(19) United States
(12) Patent Application Publication
(10) Pub. No.: US 2003/0138483 A1
(43) Pub. Date: Jul. 24, 2003

(54) Soft elastic capsules and compositions thereof

(76) Inventors: Sergio Petriconi, Anzio (IT); Alessandro Barbato, Roma (IT); Roberta Scuppa, Aprilia (IT); Azita Saleki-Gerhardt, Libertyville, IL (US); Martin Urch, Gurnee, IL (US)

Correspondence Address: STEVEN F. WEINSTOCK; ABBOTT LABORATORIES, 100 ABBOTT PARK ROAD, DEPT. 377/AP6A, ABBOTT PARK, IL, 60064-6008, US

(21) Appl. No.: 10/151,033

(22) Filed: May 17, 2002

Related U.S. Application Data
(60) Provisional application No. 60/293,623, filed on May 25, 2001.

(51) Int. Cl. A61K 9/64

(52) U.S. Cl. 424/456; 514/220

(57) ABSTRACT

A soft elastic capsule is described containing a pharmaceutical agent or a mixture of pharmaceutical agents. The soft elastic capsule is prepared from a fill composition and a shell composition that impart acceptable physical characteristics to the soft elastic capsule when in an equilibrium state. The pharmaceutical agents of the fill composition are also soluble when the soft elastic capsule is in the equilibrium state.

This application claims the benefit of U.S. Provisional Application for Patent No. 60/293,623, filed May 25, 2001.

### TECHNICAL FIELD

The invention relates to a soft elastic capsule, fill and shell compositions of the soft elastic capsule, and pharmaceutical agents contained within the soft elastic capsules. The invention further relates to soft elastic capsules and HIV protease inhibiting compounds contained in the soft elastic capsule. The soft elastic capsule can be used with a broad range of pharmaceutical agents including antibiotics, anti-AIDS pharmaceutical agents, and an array of other medicinally active agents. Important pharmaceutical agents are anti-HIV agents.

### BACKGROUND

Inhibitors of human immunodeficiency virus (HIV) protease have been approved for use in the treatment of HIV infection for several years. A particularly effective HIV protease inhibitor is (2S, 3S, 5S)-5-(N-((N-methyl-N-((2-isopropyl-4-thiazolyl)-methyl)amino)carbonyl)-L-valinyl)amino-2-(N-((5-thiazolyl)methoxy-carbonyl)-amino)-1,6 diphenyl-3-hydroxyhexane (ritonavir), which is marketed as NORVIR®. Ritonavir is known to have utility for the inhibition of HIV protease, the inhibition of HIV infection, and the enhancement of the pharmacokinetics of compounds which are metabolized by cytochrome P₄₅₀ monooxygenase. Ritonavir is particularly effective for the inhibition of HIV infection when used alone or in a combination with one or more reverse transcriptase inhibitors and/or one or more other HIV protease inhibitors. Ritonavir and processes for its preparation are disclosed in U.S. Pat. No. 5,541,206, issued Jul. 30, 1996, the disclosure of which is herein incorporated by reference. Crystalline Form II of ritonavir and processes for its preparation are disclosed in International Patent Application WO00/04016, published Jan. 27, 2000, which is incorporated herein by reference.

A HIV protease inhibitor useful in combination with ritonavir is lopinavir which has a chemical name of (2S,3S,5S)-2-(2,6-dimethylphenoxyacetyl)-amino-3-hydroxy-5-(2S-(1-tetrahydropyrimid-2-onyl)-3-methyl-butanoyl)amino-1,6-diphenylhexane. Lopinavir is an inhibitor of the HIV protease and prevents cleavage of the HIV Gag-Pol protein, resulting in the production of immature, non-infectious viral particles. The preparation of lopinavir is disclosed in U.S. Pat. No. 5,914,332, issued Jun. 22, 1999, the disclosure of which is herein incorporated by reference. Ritonavir and Lopinavir are available as a co-formulation which is marketed under the name of KALETRA®.

Factors that affect the bioavailability of a pharmaceutical agent when administered orally include aqueous solubility, pharmaceutical agent absorption, dosage strength and first pass effect. Various salts or other derivatives of the pharmaceutical agent can be prepared in attempts to achieve maximum aqueous solubility. Various capsule dosage forms can also be formulated to maximize the bioavailability of the pharmaceutical agent.

A compound of formula I (ritonavir) has been found to have good solubility in pharmaceutically acceptable organic solvents. The solubility in such solvents is enhanced in the presence of a pharmaceutically acceptable long chain fatty acid. Pharmaceutical compositions comprising HIV protease inhibitors (especially, ritonavir, lopinavir and mixtures thereof) have been prepared as a solution in a complex carrier medium comprising several components; these compositions have been described in U.S. patent application Ser. No. 09/576,097 filed May 22, 2000 and U.S. Pat. No. 6,232,333, issued May 15, 2001, both herein incorporated by reference. The carrier medium may be designed to form an emulsion upon administration thereby facilitating absorption of the HIV protease inhibitor.

There is a need for improved formulations of soft elastic capsules containing pharmaceutical agents and for procedures for manufacturing said soft elastic capsules.

### SUMMARY OF THE INVENTION

The current invention provides soft elastic capsules that have a fill, which includes pharmaceutical agent(s), an alcohol, and fatty acid; and a shell, which includes gelatin and plasticizing agent(s). The pharmaceutical agents can be, and are preferably, HIV protease-inhibiting agents. The fill and the shell of the soft elastic capsules also have an initial state, which occurs prior to the diffusion and equilibrium of fill and shell components, and an equilibrium state. In the initial state, the shell is underplasticized and the fill contains an amount of alcohol sufficient to solubilize the pharmaceutical agents and to plasticize the shell upon equilibrium. In the equilibrium state the shell is sufficiently plasticized to provide a desired degree of hardness and the pharmaceutical agents remain solubilized.

In one embodiment of the invention, the initial fill composition includes an increased amount of alcohol (relative to the amount required to solubilize the pharmaceutical agent(s)), preferably propylene glycol, HIV protease-inhibiting agent(s), a medium or long chain fatty acid, and a surfactant. The initial shell composition includes gelatin, a reduced amount of plasticizing agent(s), and water. A portion of the propylene glycol of the initial fill composition diffuses into the shell, which is underplasticized in the initial state, and plasticizes the shell to an acceptable physical condition upon equilibrium. The amount of propylene glycol remaining in the fill composition at equilibrium is sufficient to solubilize the HIV protease-inhibiting agent(s).

In another embodiment of the invention, a process for making soft elastic capsules that have a fill composition and a shell composition in which the fill composition includes pharmaceutical agent(s), is provided. In one step of the process, a fill composition is prepared that includes pharmaceutical agent(s), an increased amount of alcohol, and fatty acid. In another step, a shell composition is prepared that includes gelatin, a reduced amount of plasticizing agent(s), and water. The fill composition and the shell composition are then formed into a soft elastic capsule, in which the shell in the initial state is underplasticized. Next, the components of the fill composition and the shell composition equilibrate and the shell becomes plasticized and the pharmaceutical agent(s) remain dissolved.

### BRIEF DESCRIPTION OF THE DRAWING

FIG. 1 shows a portion of a gel encapsulation machine and a process for manufacturing capsules.

### DETAILED DESCRIPTION

As used herein ‘pharmaceutical agent’ refers to a compound with pharmacological activity.

As used herein, the term ‘soft elastic capsule’ refers to a dosage form that includes a soft shell, often partially composed of gelatin, said capsules containing a liquid fill, the fill containing a pharmaceutical agent or combination of pharmaceutical agents suitable for pharmaceutical delivery.

As used herein, the term ‘fill’ refers to the liquid composition of a soft elastic capsule, encapsulated by a shell, which contains a solubilized pharmaceutical agent or combination of pharmaceutical agents and compounds.

As used herein, the term ‘fill composition’ refers to the components, or material, in the fill. The fill composition can also indicate the concentration of materials in the fill. During the manufacturing and storage of the soft elastic capsule, the concentration of materials in the fill can change.

As used herein, the term ‘shell’ refers to the casing of the soft elastic capsule that encloses the fill.

As used herein, the term ‘shell composition’ refers to the components, or material, in the shell. The shell composition can also indicate the concentration of materials in the shell. During the manufacturing and storage of the soft elastic capsule, the concentration of materials in the shell can change.

As used herein, the term ‘initial state’ refers to the physical and chemical characteristics of the soft elastic capsule, the concentration of components in the fill, and the concentration of components in the shell, prior to the diffusion of components from the fill to the shell or from the shell to the fill. ‘Initial shell composition’ and ‘initial fill composition’ refer to the concentration of components in the fill and the concentration of components in the shell, respectively, prior to the diffusion of components from the fill to the shell or from the shell to the fill.

As used herein, the term ‘equilibrium state’ refers to the physical and chemical characteristics of the soft elastic capsule, the concentration of components in the fill, and the concentration of components in the shell, after diffusion of components from the fill to the shell or from the shell to the fill where the components have reached equilibrium in the soft elastic capsule.

As used herein, ‘acceptable physical characteristics’ of soft elastic capsules refers to the quality, hardness, disintegration time, and moisture content of the soft elastic capsules that are pharmaceutically suitable for maintaining and delivering a fill composition containing a pharmaceutical agent or combination of pharmaceutical agents.

As used herein, ‘plasticized’ refers to the relative hardness or softness of the shell of the soft elastic capsule as affected by the plasticizing agent. ‘Underplasticized’ refers to a shell composition that results in a soft elastic capsule that is too hard or brittle as a result of an inadequate amount of plasticizing agent in the shell composition.

Pharmaceutical Agents

The pharmaceutical agents of the current invention include, but are not limited to, HIV protease inhibiting compounds.

A preferred HIV protease inhibiting compound of the current invention includes a compound of formula I:

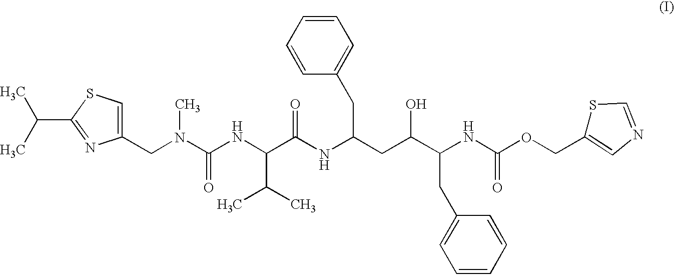

or a pharmaceutically acceptable salt thereof, disclosed in PCT Patent Application No. WO 94/14436, published Jul. 7, 1994, and U.S. Pat. No. 5,541,206, issued Jul. 30, 1996, the disclosure of both of which are herein incorporated by reference.

A preferred compound of formula I is known as ritonavir. The compounds of formula I are useful to inhibit HIV infections and, thus, are useful for the treatment of AIDS. A process for the preparation of ritonavir is disclosed in U.S. Pat. No. 5,567,823, issued Oct. 22, 1996, the disclosure of which is herein incorporated by reference. The process disclosed in this patent also produces ritonavir as crystalline Form I. Other processes for preparing ritonavir are disclosed in U.S. Pat. No. 5,491,253, issued Feb. 13, 1996; U.S. Pat. No. 6,022,989, issued Feb. 8, 2000; U.S. Pat. No. 6,160,122, issued Dec. 12, 2000; and U.S. Pat. No. 5,932,766, issued Aug. 3, 1999, all of which are incorporated herein by reference.

Pharmaceutical compositions comprising ritonavir or a pharmaceutically acceptable salt thereof are disclosed in U.S. Pat. Nos. 5,541,206, issued Jul. 30, 1996; 5,484,801, issued Jan. 16, 1996; 5,725,878, issued Mar. 10, 1998; and 5,559,158, issued Sep. 24, 1996; 6,232,333 issued May 15, 2000; and 5,948,436, issued Sep. 7, 1999, the disclosures of all of which are herein incorporated by reference.

The use of ritonavir to inhibit HIV infection is disclosed in U.S. Pat. No. 5,541,206, issued Jul. 30, 1996. The use of ritonavir in combination with one or more reverse transcriptase inhibitors to inhibit an HIV infection is disclosed in U.S. Pat. No. 5,635,523, issued Jun. 3, 1997. The use of ritonavir in combination with one or more HIV protease inhibitors to inhibit an HIV infection is disclosed in U.S. Pat. No. 5,674,882, issued Oct. 7, 1997. The use of ritonavir to enhance the pharmacokinetics of compounds metabolized by cytochrome P450 monooxygenase is disclosed in U.S. Pat. No. 6,037,157, issued Mar. 14, 2000. The disclosures of all of these patents and patent applications are herein incorporated by reference.

Another preferred HIV protease inhibiting compound of the current invention includes a compound of formula II:

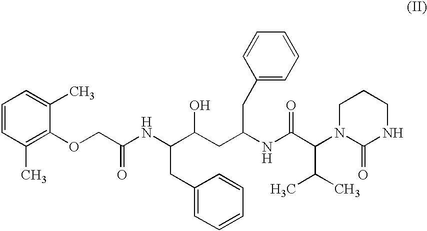

and related compounds as disclosed in U.S. Pat. No. 5,914,332 issued Jun. 22, 1999 the disclosure of which is herein incorporated by reference. A preferred compound of formula II is known as lopinavir and has a chemical name of (2S,3S,5S)-2-(2,6-dimethylphenoxyacetyl)-amino-3-hydroxy-5-(2S-(1-tetrahydropyrimid-2-onyl)-3-methyl-butanoyl)amino-1,6-diphenylhexane. The preparation of this compound is disclosed in U.S. patent application Ser. No. 5,914,332, issued Jun. 22, 1999, the disclosure of which is herein incorporated by reference.

Additional HIV protease inhibiting compounds include: N-(2(R)-hydroxy-1 (S)-indanyl)-2(R)-phenylmethyl-4(S)-hydroxy-5-(1-(4-(3-pyridylmethyl)-2(S)-N′-(t-butylcarboxamido)-piperazinyl))-pentaneamide (for example, indinavir) and related compounds, disclosed in European Patent Application No. EP 541168, published May 12, 1993, and U.S. Pat. No. 5,413,999, issued May 9, 1995, both of which are herein incorporated by reference; N-tert-butyl-decahydro-2-[2(R)-hydroxy-4-phenyl-3(S)-[[N-(2-quinolylcarbonyl)-L-asparaginyl]amino]butyl]-(4aS,8aS)-isoquinoline-3(S)-carboxamide (for example, saquinavir) and related compounds disclosed in U.S. Pat. No. 5,196,438, issued Mar. 23, 1993, which is incorporated herein by reference; 5(S)-Boc-amino-4(S)-hydroxy-6-phenyl-2(R)-phenylmethylhexanoyl-(L)-Val-(L)-Phe-morpholin-4-ylamide and related compounds, disclosed in European Patent Application No. EP532466, published Mar. 17, 1993, which is incorporated herein by reference; 1-Naphthoxyacetyl-beta-methylthio-Ala-(2S,3S)-3-amino-2-hydroxy-4-butanoyl 1,3-thiazolidine-4-t-butylamide (for example, 1-Naphthoxyacetyl-Mta-(2S,3S)-AHPBA-Thz-NH-tBU), 5-isoquinolinoxyacetyl-beta-methylthio-Ala-(2S,3S)-3-amino-2-hydroxy-4-butanoyl-1,3-thiazolidine-4-t-butylamide, and related compounds, disclosed in European Patent Application No. EP490667, published Jun. 17, 1992 and Chem. Pharm. Bull. 40 (8) 2251 (1992), which are both incorporated herein by reference; [1S-[1R*-(R*-),2S*])-N1[3-[[[(1,1-dimethylethyl)amino]carbonyl](2-methylpropyl)amino]-2-hydroxy-1-(phenylmethyl)propyl]-2-[(2-quinolinylcarbonyl)amino]-butanediamide (for example, SC-52151) and related compounds, disclosed in PCT Patent Application No. WO 92/08701, published May 29, 1992 and PCT Patent Application No. WO 93/23368, published Nov. 25, 1993, both of which are herein incorporated by reference;

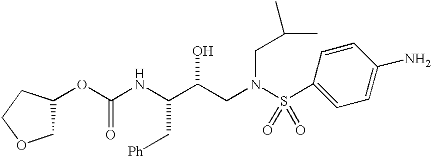

(for example, VX-478) and related compounds, disclosed in PCT Patent Application No. WO 94/05639, published Mar. 17, 1994, which is incorporated herein by reference;

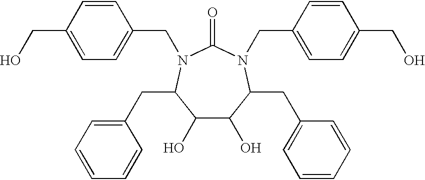

(for example, DMP-323) or

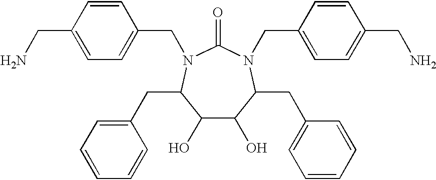

(for example, DMP-450) and related compounds, disclosed in PCT Patent Application No. WO 93/07128, published Apr. 15, 1993, which is incorporated herein by reference;

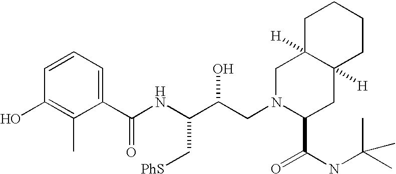

(for example, AG1343, (nelfinavir)), disclosed in PCT Patent Application No. WO 95/09843, published Apr. 13, 1995 and U.S. Pat. No. 5,484,926, issued Jan. 16, 1996, which are both incorporated herein by reference;

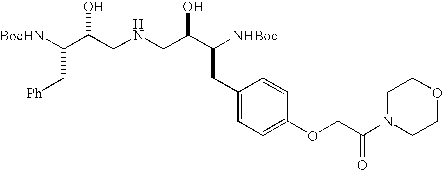

(for example, BMS 186,318) disclosed in European Patent Application No. EP580402, published Jan. 26, 1994, which is incorporated herein by reference;

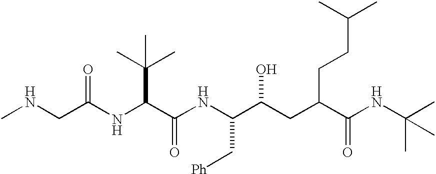

(for example, SC-55389a) and related compounds disclosed in PCT Patent Application No. WO 9506061, published Mar. 2, 1995, which is incorporated herein by reference and at the 2nd National Conference on Human Retroviruses and Related Infections, (Wash., D.C., Jan. 29-Feb. 2, 1995), Session 88; and

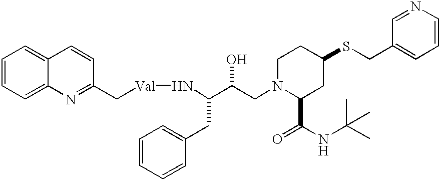

(for example, BILA 1096 BS) and related compounds disclosed in European Patent Application No. EP560268, published Sep. 15, 1993, which is incorporated herein by reference; and

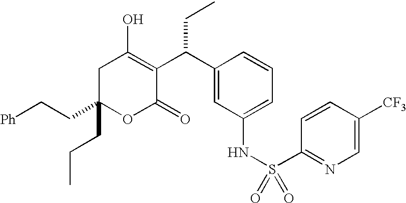

(for example, U-140690 (tipranavir)) and related compounds disclosed in PCT Patent Application No. WO 9530670, published Nov. 16, 1995, and U.S. Pat. No. 5,852,195, issued Dec. 22, 1998, the disclosures of both of which are herein incorporated by reference; and

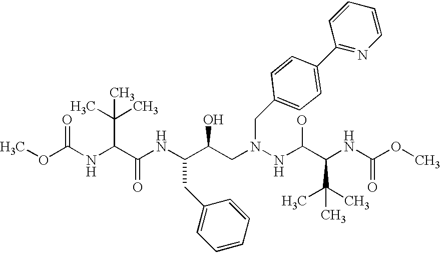

for example, BMS-232,623 (3S-(3R*, 8′R*, 9′R*, 12R*))-3,12-bis(1,1-dimethylethyl)-8-hydroxy-4,11-dioxo-9-(phenylmethyl)-6-((4-(2-pyridinyl)-phenylmethyl)-2,5,6,10,13-pentaazatetradecanedioic acid, dimethyl ester) and related compounds disclosed in International Patent Application No. WO99/36404, published Jul. 22, 1999 and International Patent Application No. WO97/40029, published Oct. 30, 1997, both herein incorporated by reference; or a pharmaceutically acceptable salt of any of the above.

Other pharmaceutical agents that can be used in the current invention include, but are not limited to, anti-viral compounds, cell growth inhibitors, antibiotics, antihistamines, analgesics, food supplements, nutrients, vitamins, steroids, and anesthetics. The current invention relates to any physiologically or pharmacologically active substance that produces a local or systemic effect.

Fill Composition

The fill composition for the soft elastic capsule of the invention can include a solubilized HIV protease inhibiting compound or a combination of solubilized HIV protease inhibiting compounds. Preferably, the HIV protease inhibiting compound is a compound of the formula I, formula II, saquinavir, nelfinavir, amprenavir, or indinavir. More preferably the compound is ritonavir, lopinavir, saquinavir, nelfinavir, amprenavir, or indinavir. Most preferably the compound is ritonavir or lopinavir. A combination of solubilized HIV protease inhibiting compounds can also be used. Preferably the combination is ritonavir or nelfinavir and another HIV protease inhibitor, for example, lopinavir, saquinavir, indinavir, amprenavir, or nelfinavir. More preferably, the combination is of ritonavir or nelfinavir and another HIV protease inhibitor, for example, lopinavir, saquinavir, indinavir, amprenavir, or nelfinavir. Most preferably the combination is ritonavir and lopinavir. A preferred ratio of the amount, as measured by weight, of ritonavir to lopinavir in the fill composition is in the range of 2:1 to 1:10; more preferably this ratio is in the range of 1:1 to 1:10; most preferably this ratio is in the range of 1:3 to 1:5, and it is most highly preferred that the ratio is about 1:4.

The HIV protease inhibiting compound or a combination of solubilized HIV protease inhibiting compounds can be present in the fill composition, as measured by weight, in a range of 10-35%. More preferably, the HIV protease inhibiting compound or compounds are present in the range of 12.5-22.5%. Most preferably, the HIV protease inhibiting compound or compounds are present in the range of 15-20%.

This HIV protease inhibiting compound or a combination of HIV protease inhibiting compounds can be solubilized in a pharmaceutically acceptable organic solvent. Preferably, the pharmaceutically acceptable organic solvent comprises from about 75% to about 90% by weight of the fill composition. More preferably, the pharmaceutically acceptable organic solvent or mixture of pharmaceutically acceptable organic solvents comprises from about 77.5% to about 87.5% by weight of the fill composition. This pharmaceutically acceptable organic solvent of the fill composition can include (1) a pharmaceutically acceptable medium and/or long chain fatty acid or mixtures thereof; (2) an alcohol, preferably propylene glycol; and (3) a pharmaceutically acceptable surfactant. Optionally, water can be included in the fill composition and can be present preferably in a percentage by weight that is relatively low as compared to the other components of the fill composition.

The fill composition can be prepared with a percentage by weight of organic solvent sufficient to solubilize the pharmaceutical agent or combination of pharmaceutical agents, for example, the HIV protease inhibitors, contained in the initial fill composition. In one embodiment of the invention, the initial fill composition contains an increased percentage by weight of alcohol, for example, propylene glycol, sufficient to solubilize the pharmaceutical agent or combination of pharmaceutical agents. The percentage of alcohol, for example, propylene glycol, is increased relative to the other components in the initial fill composition. The alcohol percentage is increased greater than the amount of alcohol necessary to solubilize the pharmaceutical agents, for example HIV protease inhibitors. In this embodiment, the propylene glycol is preferably increased by 100-200% of the minimal amount of propylene glycol necessary to solubilize the HIV protease inhibitors in the initial fill composition.

In the current invention, a portion of the amount of alcohol present in the initial fill composition will migrate into the shell of the soft elastic capsule upon equilibrium of the fill components with the shell components. A substantially smaller portion, if any, of the amounts of the other components of the fill composition, for example, the pharmaceutical agents, the fatty acid, and the surfactant, will migrate into the shell upon equilibrium. Therefore, the ranges by percentage weight in the fill composition of the pharmaceutical agents, the fatty acid, and the surfactant, as described herein, sufficiently reflect the concentration of these components of the fill composition in both the initial state and the equilibrium state.

Included in this embodiment, the increased amount of alcohol in the initial fill composition is also sufficient to retain the solubility of the pharmaceutical agent or combination of pharmaceutical agents upon equilibration of the fill composition with the shell composition. The initial fill composition is also formulated to include an amount of alcohol, for example, propylene glycol, sufficient to solubilize the pharmaceutical agent or pharmaceutical agents, for example, HIV protease inhibitors, without the addition of heat. Preferably, the initial fill composition is prepared at temperatures that avoid the degradation of a HIV protease inhibitor, for example, ritonavir.

Included in this embodiment, the increased amount of alcohol in the fill composition is also sufficient to properly plasticize the shell of the soft elastic capsule upon equilibrium of the fill composition and the shell composition. The amount of plasticizing agent in the initial shell composition is not sufficient to provide proper plasticity to the shell if no additional plasticizing agent(s), for example, alcohol, migrates into the shell during equilibrium or is otherwise added. For example, a initial shell composition formulated to be underplasticized will produce a soft elastic capsule having a shell that is too brittle if no additional alcohol, for example, polypropylene glycol, migrates into the shell. In the current invention, the excess alcohol contained in the initial fill composition can contribute to the plasticity of the shell and can become a plasticizing agent in the shell upon equilibrium.

The alcohol of the fill composition can be propylene glycol, another suitable alcohol, or mixtures thereof. Suitable alcohols include, for example, ethanol, 2-2(ethoxyethoxy)ethanol, benzyl alcohol, glycerol, polyethylene glycol 200, polyethylene glycol 300, and polyethylene glycol 400. Preferably, propylene glycol, a suitable alcohol, or a mixture thereof, is present in the initial fill composition, as measured by weight, in a range of 3-20%. More preferably, propylene glycol, a suitable alcohol, or a mixture thereof, is present in a range of 9-14%. Most preferably, propylene glycol, a suitable alcohol, or a mixture thereof, is present in the range of 10-13% in the initial fill composition. Propylene glycol can be obtained commercially from, for example Lyondell Chemie (Route Du Quai-Mineralier-Fos-S42-Mer France). Alternatively, other deriviatives of propylene glycol are available commercially and can be used in the initial fill composition, for example propylene glycol monocaprylate or propylene glycol monolaurate (Capryol® PGMC or Lauroglycol® 90, respectively, Gattefosse, Westwood, N.J.).

One component of the organic solvent of the fill composition is a pharmaceutically acceptable medium and/or long chain fatty acid or mixtures thereof. Preferably, the fatty acids are present in the fill composition, as measured by weight, in a range of 50-80%. More preferably, the fatty acids are present in a range of 62.5-75%. Most preferably, the fatty acids are present in the range of 64-70% in the fill composition. The pharmaceutically acceptable medium and/or long chain fatty acid or mixture of the fill composition can be saturated or unsaturated C₈ to C₂₄ fatty acids. These fatty acids can include, for example, Caprylic acid, Capric acid, Lauric acid, Myristic acid, Palmitic acid, Stearic acid, Behenic acid, and similar suitable medium and/or long chain fatty acid. Preferred fatty acids are mono-unsaturated C₁₆-C₂₀ fatty acids which are liquids at room temperature. A most preferred fatty acid is oleic acid, with or without additional medium and/or long chain fatty acids in the mixture. Fatty acids can be obtained commercially and one suitable source of said oleic acid is, for example, Henkel Corporation (Cincinnati, Ohio).

Another component of the organic solvent of the fill composition is a pharmaceutically acceptably surfactant. Preferably, the surfactant is present in the fill composition, as measured by weight, in a range of 0-10%. More preferably, the surfactant is present in a range of 0-7.5%. Most preferably, the surfactant is present in the range of 0-5% in the fill composition. A pharmaceutically acceptable surfactant can be a pharmaceutically acceptable non-ionic surfactant such as polyoxyethylene castor oil derivatives, for example, polyoxyethyleneglyceroltriricinoleate, polyoxyl ethylene 35 castor oil (Cremophor® EL, BASF Corp.), polyoxyethyleneglycerol oxystearate (Cremophor® RH 40 (glycerol polyethyleneglycol oxystearate) or Cremophor® RH 60 (polyethyleneglycol 60 hydrogenated castor oil), BASF Corp., and the like), block copolymers of ethylene oxide and propylene oxide, also known as polyoxyethylene polyoxypropylene block copolymers or polyoxyethylenepolypropylene glycol, for example Poloxamer® 124, Poloxamer® 188, Poloxamer® 237, Poloxamer® 338, Poloxamer® 407, and the like, (BASF Wyandotte Corp.), a mono fatty acid ester of polyoxyethylene (20) sorbitan (for example, polyoxyethylene (20) sorbitan monooleate (Tween® 80), polyoxyethylene (20) sorbitan monostearate (Tween® 60), polyoxyethylene (20) sorbitan monopalmitate (Tweent 40), polyoxyethylene (20) sorbitan monolaurate (Tween® 20)) and the like), or a sorbitan fatty acid ester (including sorbitan laurate, sorbitan oleate, sorbitan palmitate, sorbitan stearate and the like). A preferred pharmaceutically acceptable surfactant is polyoxyl 35 castor oil (Cremophor® EL, BASF Corp.), polyoxyethylene (20) sorbitan monolaurate (Tween® 20), polyoxyethylene (20) sorbitan monooleate (Tween(t 80) or a sorbitan fatty acid ester, for example sorbitan oleate. A most preferred pharmaceutically acceptable surfactant is polyoxyl 35 castor oil (Cremophor® EL, BASF Corp.).

Optionally, the fill composition can include water. If water is included in the fill composition it is preferably present in a percentage by weight that is relatively low as compared to the other components of the fill composition. Preferably the percentage of water in the fill composition is no greater than 3% by weight and more preferably no greater than 1.5% by weight of the fill composition.

In addition, the composition of the invention can comprise antioxidants (for example, ascorbic acid, BHA (butylated hydroxyanisole), BHT (butylated hydroxytoluene), vitamin E, and the like) for chemical stability.

Solutions as described herein can include micellar solutions, which are thermodynamically stable systems formed spontaneously in water above a critical temperature and concentration. Micellar solutions contain small colloidal aggregates (micelles), the molecules of which are in rapid thermodynamic equilibrium with a measurable concentration of monomers. Micellar solutions exhibit solubilization phenomena and thermodynamic stability.

Optionally, the fill composition can include a pharmaceutically acceptable acid. Pharmaceutically acceptable acid as used herein can include (i) an inorganic acid such as hydrochloric acid, hydrobromic acid, hydroiodic acid and the like, (ii) an organic mono-, di- or tri- carboxylic acid (for example, formic acid, acetic acid, adipic acid, alginic acid, citric acid, ascorbic acid, aspartic acid, benzoic acid, butyric acid, camphoric acid, gluconic acid, glucuronic acid, galactaronic acid, glutamic acid, heptanoic acid, hexanoic acid, fumaric acid, lactic acid, lactobionic acid, malonic acid, maleic acid, nicotinic acid, oxalic acid, pamoic acid, pectinic acid, 3-phenylpropionic acid, picric acid, pivalic acid, propionic acid, succinic acid, tartaric acid, undecanoic acid and the like) or (iii) a sulfonic acid (for example, benzenesulfonic acid, sodium bisulfate, sulfuric acid, camphorsulfonic acid, dodecylsulfonic acid, ethanesulfonic acid, methanesulfonic acid, isethionic acid, naphthalenesulfonic acid, p-toluenesulfonic acid and the like).

The fill composition can also contain other pharmaceutical agents, for example, anti-viral compounds, cell growth inhibitors, antibiotics, antihistamines, analgesics, food supplements, nutrients, vitamins, steroids, or anesthetics. Other pharmaceutical agents can be co-administered with the HIV protease inhibitors, if compatible with the inhibitors, or by themselves.

Shell Composition

The shell composition of the current invention is suitable for encapsulating fill compositions including a pharmaceutically active compound or a mixture of pharmaceutically active compounds, for example HIV protease inhibitors, as described herein. The formulation of the initial shell composition is particularly suitable for maintaining the solubility of the components of the fill composition after equilibrium has been established between components of the fill composition and the shell composition. In addition, the current invention provides components of the initial shell composition and a process utilizing these components for the manufacture of a soft elastic capsule containing pharmaceutically active compound(s), as described herein, wherein the shell of the soft elastic capsule acquires and retains desirable physical characteristics during and after the manufacturing of the soft elastic capsule.

In one embodiment of the current invention, the initial shell composition is formulated to produce, in the absence of any later added or migrated plasticizing agents, a shell that contains a percentage by weight of a plasticizing agent or agents that is too low to provide appropriate hardness. In the composition of this invention, the initial shell is underplasticized and upon equilibrium of the shell composition with the fill composition, as described herein, the shell becomes plasticized to an acceptable physical range. The increased percentage by weight of alcohol, for example, propylene glycol, of the initial fill composition contributes to increasing the plasticity of the shell upon equilibrium of the fill and shell components. Upon reaching equilibrium, the alcohol of the fill composition, for example, propylene glycol, which has migrated into the shell, acts as a plasticizing agent. Upon reaching equilibrium the shell has acceptable physical properties, for example, hardness, and the fill composition maintains the solubility of the pharmaceutical agent or pharmaceutical agents, for example HIV protease inhibitors, within the fill.

The components of the initial shell composition (i.e., prior to capsule formation or encapsulation of the fill composition) of the current invention include gelatin, a plasticizing agent, preferably Anidrisorb™, or optionally glycerin, and water. Optionally, the shell composition can also include a pigment, for example titanium dioxide, and optionally a dye, for example FD&C yellow number 6.

During the preparation of the soft elastic capsule the initial shell composition is prepared as a gelatin solution that contains water. During the preparation the soft elastic capsule the gelatin solution is heated and typically looses an amount of water. The formulations of the initial shell composition of the current invention include water; however, it is understood that water is lost during the manufacturing and drying processes. These losses will change the amount of water in the shell as the soft elastic capsule reaches an equilibrium state. Additionally, components in the initial fill composition will migrate into the shell composition thereby also changing the composition of the shell as the soft elastic capsule reaches an equilibrium state. The formulations listed herein refer to an initial shell composition, prior to loss of water or migration of fill components into the shell, unless otherwise noted.

Gelatin can be obtained from skin and bone of bovine and porcine sources. Acid or basic preparations of gelatin can be used as an ingredient in the initial shell composition. Depending on the source and the process of preparing the gelatin, different levels of gelatin strength can be used in the initial shell composition. Preparation of bovine gelatin, for example, 195B acid, can be used in the shell composition. Bovine gelatin can be obtained commercially from, for example, SKW Biosystems (Cedex, France). Gelatin can be present in the shell composition, by weight, preferably in the range of 30-60%, more preferably in the range of 35-55%, and most preferably in the range of 40-50%.

The plasticizing agent of the initial shell composition can be sorbitol, sorbitan, glycerol, xylitol, polyglycerol, propylene glycol, glucose, fructose, glucose, polyols, for example, macrogol 400, 600, 1200, 1500, 2000, or 4000, macrogols between 400 and 4000 (available from, for example, Union Carbide (Noorderlaan, 147 2030 Anversa Belgio) or Poloxamers. Preferably, a mixture of sorbitol, sorbitans, mannitol, and hydrogenated saccharides, which is available as a composition commercially available under the trade name of Anidrisorb™, is used as the plasticizing agent. Anidrisorb™ can be obtained commercially from Roquette Freres (Letrem, France). The shell composition can also have a combination of more than one plasticizing agent. For example, Anidrisorb™ can be used in combination with other plasticizing agents, for example, glycerol. A preferred combination of plasticizing agents contains any of the following reagents: Anidrisorb™, glycerol, and macrogols.

In the present invention, the initial shell composition has a relatively low percentage of plasticizing agent or combination of plasticizing agents, for example, not more than 18%, by weight. Preferably the plasticizing agent or agents is present in a range of 4-18% in the initial shell composition, more preferably in the range of 8-17%, and most preferably in the range of 10-16%.

In a preferred embodiment of the current invention, either Anidrisorb™ or a combination of Anidrisorb™ and glycerin is used as the plasticizing agent in the initial shell composition. If a combination of Anidrisorb™ and glycerin are used, the ratio of Anidrisorb™ to glycerin is preferably in the range of 12:1 to 2:1.

The initial shell composition also includes water as a component. It is understood that water is used in the formulation of the initial shell composition and that water is eliminated from the shell during the manufacturing process of the soft elastic capsule. Water can be present in the shell composition, by weight, in a range of 20-55%, more preferably in the range of 25-50%, and most preferably in the range of 30-45%.

The initial shell composition of the current invention can also be defined by the ratio of gelatin to plasticizing agent. Preferably the ratio of gelatin to plasticizing agent in the initial shell composition is in the range of 2:1 to 10:1, more preferably in the range of 2.3:1 to 7:1, and most preferably in the range of 2.5:1 to 5:1.

In one embodiment of the invention, the initial shell composition includes gelatin, by weight, in the range of 25-60%, a plasticizing agent, or combination of plasticizing agents in the range of 4-18%, and water in a range of 20-55%. More preferably, the initial shell composition includes gelatin, by weight, in the range of 30-55%, a plasticizing agent, or combination of plasticizing agents in the range of 8-17%, and water in a range of 25-50%. Most preferably, the initial shell composition includes gelatin, by weight, in the range of 35-50%, a plasticizing agent, or combination of plasticizing agents in the range of 10-16%, and water in a range of 30-45%.

The soft elastic gelatin capsule material can also comprise additives such as, opacifiers, dyes or excipients, for example, flavors, sweeteners, and preservatives.

The current invention provides a soft elastic capsule that, after equilibrium of the fill composition and shell composition, has acceptable physical properties and maintains the active ingredients, for example, HIV protease inhibitor(s), in a soluble state. One aspect of the soft elastic capsule in the equilibrium state is the ratio of gelatin to plasticizing agent, which can affect the physical characteristics of the soft elastic capsule. In a preferred embodiment of the invention, the gelatin to plasticizing agent ratio is in the range of 2:1 to 4:1 when the shell and fill composition are in the equilibrium states. This range of gelatin to plasticizing agent can provide the soft elastic capsule with acceptable physical properties.

An alcohol partition coefficient can be determined after the soft elastic capsule has reached an equilibrium state. The alcohol partition coefficient, for example, the propylene glycol partition coefficient, is a ratio reflecting the amount of propylene glycol in the shell at equilibrium compared to the amount of propylene glycol in the fill in the initial state, respectively. In one embodiment of the invention, the propylene glycol partition coefficient between the shell at equilibrium and the fill in the initial state is preferably in the range of 1:2-1:2.5, respectively, and more preferably in the range of 1:2.2-1:2.4, respectively.

Various manufacturing processes that are well known in the art can be used to form soft elastic capsules from the fill and shell compositions described herein. Descriptions of these processes can be found in various references, for example, Principles and Practices of Pharmaceutics, The Pharmaceutical Codex, Twelfth Edition.; Ed.: Walter Lund, The Pharmaceutical Press, London, 1994, p23-24 or Oral Solid Dosage Forms, Remington's Pharmaceutical Sciences, 17th Edition; Ed.: Gennaro, A. R., Mack Publishing Co., Easton, Pa., 1985, p.1629-1631. These various processes include, for example, a seamless capsule method, microencapsulation, and soft gelatin coating of tablets. Various manufacturing machines can be used for manufacturing the capsules, for example, a Norton® Capsule Machine, Accogel® Capsule Machine, or a Liner® machine.

A preferred process for manufacturing soft elastic capsules from fill and shell compositions, as described herein, is performed according to the Rotary Die Process. Generally, two continuous gelatin ribbons, which are formed by the capsule-forming machine, are brought together between a pair of revolving dyes and an injection wedge. Insertion of the fill composition under pressure and sealing of the capsule shell occur simultaneously and the two processes are precisely coordinated. The process of sealing also separates the formed capsules. The Rotary Die machine can also consist of multiple injection pumps allowing multiple capsules to be prepared at once. Accuracy of injection of the proper amount of fill solution and capsule weight are periodically determined by analytical balances associated with the machine.

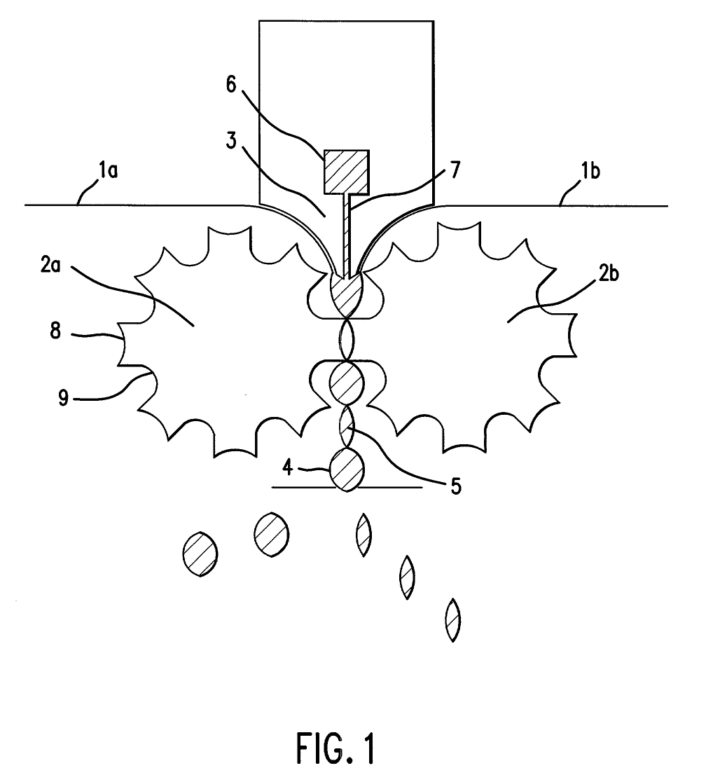

According to FIG. 1, the mechanical workings of a Rotary Die machine are shown. Gel ribbons 1a and 1b are fed onto rotary cylinders 2a and 2b, respectively, where, as shown in FIG. 1, rotary cylinder 2a rotates clockwise and rotary cylinder 2b rotates counterclockwise. Both rotary cylinders contain sprocket portions 8 and divit portions 9. Gel ribbons 1a and 1b are drawn between rotary cylinders 2a and 2b and injection wedge 3 by the rotary motion of the cylinders. Injection pump 6 of injection wedge 3 pumps a predetermined amount of a fill composition 7 between gel ribbons 1a and 1b while cylinders rotate. Capsule 4 is formed by opposing divit portions 9 of rotary cylinders 2a and 2b as fill composition 7 is injected between gel ribbons 1a and 1b. Consecutive capsules 4 emerge from the rotary cylinders 2a and 2b separated by a gelatin spacer 5.

Typically, soft elastic capsules are dried following their formation. Drying processes can include tumble drying, or successive tumble drying, and sheet or tray drying. Capsules can be tumble dried for various lengths of time, for example, from 0 to 6 hours and at various temperatures, for example, from 20-25° C. Preferably, the capsules of the current invention are tumble dried for a total of 0.5 to 3 hours and at a temperature in the range of 20-25° C.

Physical and Chemical Characteristics of soft elastic Capsule Following Manufacture

The fill composition and shell composition of the present invention allow a soft elastic capsule containing a pharmaceutical agent or combination of pharmaceutical agents to be manufactured that has acceptable physical and chemical characteristics. The soft elastic capsule can be manufactured to have acceptable physical and chemical characteristics or these characteristics can be acquired as the soft gel capsule reaches an equilibrium state. The equilibrium state can be achieved, at least partially, by the diffusion of an alcohol, for example, propylene glycol, into the shell. Following manufacture, acceptable physical characteristics include capsules having a shell that has acceptable plasticity (as measured by hardness), a shell that has an acceptable disintegration time, and a fill that has an acceptable moisture content. Desirable chemical characteristics include capsules having a fill that has an acceptable alcohol, for example, propylene glycol, and water content; the alcohol, for example, propylene glycol, content is important in the solubility of the pharmaceutical agent or combination of pharmaceutical agents in the fill composition.

The physical characteristics of the shell can change during the manufacturing process. Particularly, during the steps of drying of the soft elastic capsule, the propylene glycol of the fill migrates into the shell and increases the plasticity of the shell. The current invention provides fill compositions and shell compositions that can be used to produce a soft elastic capsule that has an acceptable level of plasticity following manufacturing and drying.

Plasticity of the shell of the manufactured capsule can be measured by its hardness initially after manufacturing and over a longer period of time, for example, over a period of months or years. Hardness of the shell capsule can be measured in Newtons, using a hardness tester, for example a Bareiss™ U73 hardness tester (Oberdischingen, Germany) can be used to test the capsules according to manufacturer's instructions and accepted standards.

Upon initial manufacture, the capsules preferably have a hardness in the range from about 6 to about 12 Newtons, more preferably in the range from about 7 to about 111 Newtons, and most preferably in the range from about 7.25 to about 10.75 Newtons. After drying, and over a period of time, the hardness of the soft elastic capsule slowly decreases and then stabilizes and the propylene glycol concentration in the fill and in the shell reach an equilibrium state. Preferably, over a period of two weeks to three years the hardness of the gel capsule is maintained in a range from about 4.5 to about 12 Newtons, more preferably in a range from about 4.75 to about 12 Newtons, and most preferably in a range from about 5 to about 10 Newtons.

Upon initial manufacture, and over a longer period of time and after the equilibrium state is reached, for example, over a period of months or years, the soft elastic capsule can be physically examined wherein the total weight, the fill weight, and shell weight can be determined. Such physical examinations can be conducted according to accepted standards. Shell weight can be determined by emptying capsules of the fill and washing the shell with an organic solvent to remove residual fill. Suitable organic solvents for capsule washing include, for example, methylene chloride and chloroform. The weight of the fill is calculated by the difference between the weight of the shell and the total weight of the capsule. Preferably, over a period of two weeks to three years the total weight of the gel capsule deviates not more than 5% from the initial total weight of the gel capsule, more preferably not more than 2.5% and most preferably not more than 1%. Preferably, over a period of two weeks to three years the total weight of the fill of the gel capsule deviates not more than 15% from the original fill weight of the capsule, more preferably not more than 10% and most preferably not more than 7.5%.

Moisture in the fill of the gel capsule can be determined using a suitable apparatus, for example, a titrator. A suitable titrator is a Karl Fischer titrator apparatus, used according to the manufacturer's directions and by accepted standards.

Preferably, over a period of two weeks to three years the total moisture of the fill of the gel capsule is not more than 2% of the fill weight of the gel capsule, more preferably not more than 1.5% and most preferably not more than 1%.

Disintegration time of the capsule can be measured by accepted standards using, for example, a disintegration tester. Preferably, over a period of two weeks to three years the disintegration time of the gel capsule remains not more than 30 minutes according to this procedure.

Propylene glycol content in the fill can be measured by accepted standards using, for example, a gas chromatography technique.

After equilibration the fill can have a propylene glycol concentration at 5° C. of approximately 40-50 mg per gram of fill. This concentration of propylene glycol is sufficient to maintain the solubility of the pharmaceutical agent or pharmaceutical agent mixture. Preferably, over a period of two weeks to three years the propylene glycol concentration of the fill of the gel capsule is at least 40 mg per gram of fill, more preferably at least 45 mg per gram of fill and most preferably at least 50 mg per gram of fill.

### EXAMPLE 1

### Soft Elastic Capsule Containing Ritonavir/Lopinavir Mixture

Fill compositions were prepared in a Pilot® Mixing Vessel 30L Code 730 (Pharmagel, Lodi, Italy). The mixer was operated at about 1400 R.P.M. during working conditions unless otherwise noted. During working conditions the pressure inside the vessel was maintained at 1 bar (ambient conditions).

Materials for the fill composition were obtained as follows: oleic acid (Medinique) was obtained from Henkel Corporation (Cincinnati, Ohio); propylene glycol (EP) was obtained from Hulls (Lyondell chemie, Route Du Quai, Mineralier, Fos-S42-Mer France); and Cremophor® EL (polyoxyl 35 castor oil), a surfactant, was obtained from BASF Corp. (Worcester, Mass., USA). The synthesis of ritonavir is disclosed in U.S. Pat. No. 5,541,206, issued Jul. 30, 1996, the disclosure of which is herein incorporated by reference. The synthesis of ritonavir crystal form II is disclosed in International Patent Application No. WO00/04016, published Jan. 27, 2000, the disclosure of which is herein incorporated by reference. The synthesis of lopinavir is disclosed in U.S. Patent Application No. 5,914,332, issued Jun. 22, 1999, the disclosure of which is herein incorporated by reference.

Table 1 indicates the amounts of compounds of the fill composition as measured in milligrams per gram of the fill composition. Two different preparation of fill composition, PS-A and PS-B, are shown for this example.

TABLE 1
Composition of Fill

| | Batch: PS-A | | Batch: PS-B | |
|---|---|---|---|---|
| | | Capsules | | Capsules |
| | Fill (mg/g) | (mg/cap) | Fill (mg/g) | (mg/cap) |
| Formulation | | | | |
| Oleic Acid | 685.11 | 602.9 | 662.53 | 602.9 |
| Propylene Glycol (PG) | 101.25 | 89.1 | 130.88 | 119.1 |
| Cremophor ® EL | 24.32 | 21.4 | 23.52 | 21.4 |
| Ritonavir | 37.84 | 33.3 | 36.59 | 33.3 |
| Lopinavir | 151.48 | 133.3 | 146.48 | 133.3 |
| General | | | | |
| Fill Weight | | 880 | | 910 |
| Fill Volume (μL) | | 938 | | 967 |

For this preparation, 4.5 Kg of fill PS-A and 4.6 Kg PS-B were prepared.

For batch PS-A, 3.083 Kg of oleic acid, 455.6 g of Propylene Glycol, 109.4 g of Cremophor® EL, 170.3 g of Ritonavir, and 681.7 g of Lopinavir were used.

For batch PS-B, 3.048 Kg of oleic acid, 602 g of propylene glycol, 108.2 g of Cremophor® EL, 168.3 g of ritonavir, and 673.8 g of lopinavir were used.

First, the oleic acid was added to the tank. Propylene glycol was then added to the tank and mixed for five minutes. Next, ritonavir was added and mixed until completely dissolved. Next, lopinavir was added and mixed until completely dissolved. Finally, Cremophor™ EL was added to the clear solution which was mixed for 10 minutes. The product was kept under a nitrogen atmosphere.

Gelatin compositions were prepared in a Pilot® Melter 100 L Code 0204 Code (Pharmagel, Lodi, Italy). The stirring speed was maintained in a range of 25-35 R.P.M. during working conditions unless otherwise noted. During working conditions the pressure inside the vessel was at 1 bar (ambient conditions).

Anidrisorb™ 85/70 (CAS n°: 50-70-4/12441-09-7 EINECS:200-061-5/235-671-O) was obtained from Roquette Freres (Letrem, France); Gelatin 195 Bloom Acid was obtained from SKW Biosystem (84808 Isle Sur La Sourge, Cedex, France); titanium dioxide E171 was obtained from Anstead International (Radford Way, Billericay, Essex CM12 ODE, England); Dye Yellow F.D.& C. No. 6 was obtained from F.11i Fiorio-Colori S.P.A. (Via Italia, 28-20060 Gessate (MI)).

Table 2 indicates the percentage weight of compounds of the shell composition prior to heating the shell composition. During the preparation of the shell and the manufacturing of the soft elastic capsules water was eliminated.

TABLE 2
Composition of Gelatin Base (shell)

| | Batch: BA |
|---|---|
| Shell Composition | (%) |
| Anidrisorb | 10 |
| Gelatin | 50 |
| Titanium Dioxide | 0.26 |
| Dye Yellow 6 | 0.14 |
| Water | 39.6 |

For this preparation (shell composition BA), the batch size of the shell composition was 40 Kg.

20.84 Kg water (includes 5.0 Kg excess to account for water loss during heating) and 3.9 Kg of Anidrisorb™ 85/70 was loaded into the melter. The solution was then heated to 80° C. with mixing (heating to 80° C. took approximately 30 minutes). Next, 104 g of titanium dioxide and 56 g of yellow dye FD&C No. 6 was mixed with approximately 100 g of Anidrisorb™ 85/70 using a Silverson L4R to obtain a homogenous dispersion. When the solution reached 80° C., 20 Kg of gelatin was added and mixing was maintained under a vacuum. The temperature was maintained at 80° C., the vacuum at less than 0.1 bar, and the mixing rate at 22 and 44 R.P.M. (using two mixing blades, respectively, rotating in opposite directions) until 5.0 Kg of water was removed from the mixing chamber.

Encapsulation was carried out using a MK3LDS Encapsulation Line (Pharmagel, Lodi, Italy) which includes: Encapsulation Machine, Code 320; Complete Set of Die Roll, Code 950, Capsule Conveyor, Code 322; Electrical Control Panel, Code 335; and Tumble Drier, Code 323. For preparation of soft elastic capsules the following manufacturing conditions were used unless otherwise noted: the transfer line temperature was set at 60° C.; the spread box temperature was set in the range of 54-58° C.; the machine speed was set at 3 R.P.M. (die roll speed), the drum temperature was set in the range of 14-16° C.; the injection segment (wedge) temperature was set at 42-44° C.; and the gelatin thickness was set in the range of 37-39 thousandths of an inch. The die roll was 15 oblong S. The lubrication used was Migliol 812™ (Dyna-France)/migliol 812 and lethicin. The encapsulation procedure is carried out at room temperature, or approximately 20-25° C., and in a relative humidity of approximately 15%.

The portable tank of the encapsulation machine was preheated to 60° C. and maintained at 60° C. for the entire encapsulation process. After the portable tank reached 60° C., the shell composition (gelatin base), following preparation, was transferred from the melter to the portable tank of the encapsulation machine. The gelatin base transfer tank was then connected to the encapsulation line through the gelatin cleanline pump. Next, the machine temperature and speed was set. The fill solution was then placed in the encapsulation machine hopper and the encapsulation thickness set. Encapsulation of the fill solution was then started.

Tumble drying of the soft elastic capsules emerging from the encapsulation machine took place in tumble driers #1, #2, and #3, successively, each for 30 minutes each with drying at 20-25° C. All three tumble driers were connected in series. Following tumble drying the capsules were then spread on trays and placed on a trolley. The trolley was placed in a dry tunnel at 20-25° C. for approximately 48 hours. The tray drying time for different batches of capsules may vary in the range of 36 to 72 hours depending on the physical condition of the capsules.

Capsules were stored at either 2-8° C. (no humidity control) or at 25° C. (with 60% relative humidity control) for physical stability assays.

A representative sample of soft elastic capsules was physically examined to determine the total weight, the fill weight, and the shell weight. Total weight was determined weighing the individual capsules with a standard analytical scale. Shell weight was determined by emptying capsules of the fill and washing the shell with chloroform to remove residual fill. The weight of the fill was calculated with a standard analytical scale by the difference between the weight of the shell and the total weight of the same capsule. The sealing area was verified with a microscope observed at 50× magnification and was calculated by dividing the thickness of the shell in the sealing area divided by the thickness of the shell in an area of the shell away from the sealing area. The value of the sealing area is typically shown as a percentage.

The moisture content of the fill of the soft elastic capsules was determined using a Mettler Toledo DL-38 Karl Fischer Titrator according to manufacturer's instructions. In the titration flask sufficient methanol was added to immerse the double platinum electrode and titration was performed to the end point. Standardization was performed using water. Using a 10 mL disposable syringe and needle, the fill content of 10 capsules was withdrawn. Approximately one third of the fill volume withdrawn from the capsules was transferred to the titration vessel and titration was performed to the end point and the volume added was recorded. The percentage moisture was calculated by dividing the titrating reagent per sample by the sample weight (mg) and multiplying by the water equivalent and 100.

Hardness of the capsule shell was measured in Newtons using a BAREISS hardness tester model G7394A (Prüfgerätebau GmbH, D-89610 Oberdischingen, Germany) according to manufacturer's instructions.

Disintegration time for capsules were tested in water at 37±1° C. using an EP/USP disintegration tester DT3 (Sotax, Basel, Switzerland) consisting of a basket-rack assembly, a 1000 ml low-form beaker for the immersion fluid, and a thermostatic arrangement for heating the fluid to the temperature designated. The disintegration tester was operated according to manufacturer's instructions.

Propylene glycol content was determined using capillary gas chromatography. The contents of ten capsules were emptied into a glass container and weighed. About 2 g of the capsule contents were placed into a 100 mL volumetric flask and 5.0 mL of an internal standard solution (butanol diluted with methanol) was added, and then additionally diluted with methanol and mixed. 1 mL of each sample was injected using an injection device into a fused silica wall coated open tubular capillary column (DB-Wax, J& W Scientific) using helium as a carrier gas. The injector temperature was 185° C. and the detector temperature was 220° C. The helium had a flow rate of approximately 4.5 mL/min. A gas chromatograph equipped with split injector and flame ionization detector was used for detection and the run time was approximately 34 minutes.

TABLE 3
Physical data for the soft elastic capsules made
from fill batch PS-A and shell batch BA.

| | Capsules | Shell | Fill | |
|---|---|---|---|---|
| | weight | weight | weight | Hardness |
| Sample description | (mg) | (mg) | (mg) | (N) |
| Initial (in process) | 1528 | 650 | 878 | nt |
| 24 hours in tray dryer | 1312 | 439 | 873 | 10 |
| 48 hours in tray dryer | 1290 | 435 | 855 | 11 |

nt = not tested

TABLE 4
Physical data for the soft elastic capsules made
from fill batch PS-B and shell batch BA.

| | Capsules | Shell | Fill | |
|---|---|---|---|---|
| | weight | weight | weight | Hardness |
| Sample description | (mg) | (mg) | (mg) | (N) |
| Initial (in process) | 1543 | 646 | 897 | nt |
| 24 hours in tray dryer | 1319 | 455 | 864 | 9 |
| 48 hours in tray dryer | 1260 | 386 | 874 | 10 |

TABLE 5
Physical data (stablility) for the soft elastic capsules made
from fill batch PS-A and shell batch BA.

| Test | Initial | 4 Wks 5° C. |
|---|---|---|
| Total Weight (mg) | 1289 | 1287 |
| Shell Weight (mg) | 412 | 415 |
| Fill Weight (mg) | 877 | 872 |
| Hardness (Newton) | 11.3 | Nt |
| Fill Moisture (mg/g) | 11.4 | 10.4 |
| PG assay (mg/g) | 52.3 | 46.0 |
| Disintegration Time (min) | 10 | 11 |

TABLE 6
Physical data (stablility) for the soft elastic capsules made
from fill batch PS-B and shell batch BA.

| Test | Initial | 4 Wks 5° C. |
|---|---|---|
| Total Weight (mg) | 1292 | 1295 |
| Shell Weight (mg) | 427 | 433 |
| Fill Weight (mg) | 864 | 862 |
| Hardness (Newton) | 9.8 | Nt |
| Fill Moisture (mg/g) | 11.5 | 10.8 |
| PG assay (mg/g) | 60.2 | 48.7 |
| Disintegration Time (min) | 10 | 11 |

### EXAMPLE 2

### Soft Elastic Capsule Containing Ritonavir/Lopinavir Mixture

The fill composition was prepared using the same equipment and conditions as described in Example 1. Materials for the fill composition were the same as described in Example 1. The percentage of each component by weight was similar to that of Batch PS-B as described in Example 1.

TABLE 7
Composition of Fill

| | Fill (mg/g) | Capsules (mg/cap) |
|---|---|---|
| Formulation | | |
| Oleic Acid | 662.53 | 602.9 |
| Propylene Glycol (PG) | 130.88 | 119.1 |
| Cremophor EL | 23.52 | 21.4 |
| Ritonavir | 36.59 | 33.3 |
| Lopinavir | 146.48 | 133.3 |
| General | | |
| Fill Weight | | 910 |

Gelatin compositions were prepared using the same equipment and conditions as described in Example 1

Materials for the shell composition were the same as described in Example 1 with the exception of the addition of glycerin to shell formulations BE, BF, and BG. Table 2 indicates the percentage weight of compounds of the shell composition prior to heating the shell composition. It is understood that during the preparation of the shell and the manufacturing of the soft elastic capsules water is eliminated.

TABLE 8
Compositions of Gelatin Base (shell)

Batches:

| | BB | | | | | |
|---|---|---|---|---|---|---|
| Gelatin base Composition | % | BC | BD | BE | BF | BG* |
| Anidrisorb | 12 | 14 | 16 | 12 | 12 | 12 |
| Glycerin | 0 | 0 | 0 | 2 | 4 | 2 |
| Gelatin | 47.5 | 47.5 | 47.5 | 47.5 | 47.5 | 47.5 |
| Titanium Dioxide | 0.26 | 0.26 | 0.26 | 0.26 | 0.26 | 0.26 |
| Dye Yellow 6 | 0.14 | 0.14 | 0.14 | 0.14 | 0.14 | 0.14 |
| Water | 40.1 | 38.1 | 36.1 | 38.1 | 36.1 | 38.1 |

*Formulation BG contains 50% bloom gelatin 165 and 50% bloom gelatin 195.

Shell compositions were prepared using the same equipment and conditions as described in Example 1.

Manufacturing and drying of the soft elastic capsules was carried out using the same equipment and conditions as described in Example 1. Physical and chemical testing of the soft elastic capsules, including the fill and the shell were the same as described in Example 1 with the exception that the testing was conducted over a 52 week time period.

TABLE 9
26 weeks stability data for batch BB (lot 60276N6)

| Test | Initial | 26 Wks 5° C. |
|---|---|---|
| Total Weight (mg) | 1269 | 1280 |
| Shell Weight (mg) | 374 | 394 |
| Fill Weight (mg) | 896 | 886 |
| Hardness (Newton) | 9.5 | 6.6 |
| Fill Moisture (mg/g) | 5.2 | 6.8 |
| PG assay (mg/g) | 29.5 | 54.8 |
| Disintegration Time (min) | 9 | 11 |

TABLE 10
52 weeks stability data for batch BC (lot 60277N6)

| Test | Initial | 26 Wks 5° C. | 52 Wks 5° C. |
|---|---|---|---|
| Total Weight (mg) | 1274 | 1276 | 1277 |
| Shell Weight (mg) | 393 | 410 | 409 |
| Fill Weight (mg) | 881 | 865 | 868 |
| Hardness (Newton) | 10 | 6.1 | 6.9 |
| Fill Moisture (mg/g) | 5.5 | 6.8 | 6.8 |
| PG assay (mg/g) | 58.3 | 56.1 | 58.9 |
| Disintegration Time (min) | 8 | 11 | 11 |

TABLE 11
26 weeks stability data for batch BD (lot 60278N6)

| Test | Initial | 26 Wks 5° C. |
|---|---|---|
| Total Weight (mg) | 1267 | 1280 |
| Shell Weight (mg) | 409 | 431 |
| Fill Weight (mg) | 858 | 849 |
| Hardness (Newton) | 9.2 | 5.9 |
| Fill Moisture (mg/g) | 4.9 | 5.9 |
| PG assay (mg/g) | 54.4 | 52.6 |
| Disintegration Time (min) | 9 | 11 |

TABLE 12
26 weeks stability data for batch BE (lot 60279N6)

| Test | Initial | 26 Wks 5° C. |
|---|---|---|
| Total Weight (mg) | 1259 | 1266 |
| Shell Weight (mg) | 397 | 413 |
| Fill Weight (mg) | 862 | 852 |
| Hardness (Newton) | 7.8 | 5.1 |
| Fill Moisture (mg/g) | 5.1 | 4.5 |
| PG assay (mg/g) | 54.0 | 51.5 |
| Disintegration Time (min) | 9 | 9 |

TABLE 13
26 weeks stability data for batch BF (lot 60280N6)

| Test | Initial | 26 Wks 5° C. |
|---|---|---|
| Total Weight (mg) | 1249 | 1262 |
| Shell Weight (mg) | 384 | 407 |
| Fill Weight (mg) | 864 | 854 |
| Hardness (Newton) | 9.1 | 7.1 |
| Fill Moisture (mg/g) | 5.2 | 4.7 |
| PG assay (mg/g) | 56.0 | 54.1 |
| Disintegration Time (min) | 11 | 10 |

TABLE 14
52 weeks stability data for batch BG (lot 60281N6)

| Test | Initial | 26 Wks 5° C. | 52 Wks 5° C. |
|---|---|---|---|
| Total Weight (mg) | 1233 | 1244 | 1229 |
| Shell Weight (mg) | 368 | 387 | 366 |
| Fill Weight (mg) | 866 | 857 | 864 |
| Hardness (Newton) | 8.9 | 6.3 | 7.4 |
| Fill Moisture (mg/g) | 4.9 | 5.8 | 5.9 |
| PG assay (mg/g) | 59.2 | 58.7 | 55.6 |
| Disintegration Time (min) | 10 | 10 | 11 |

We claim:

1. A soft elastic capsule having a fill and a shell, the fill comprising:
a pharmaceutical agent or combination of pharmaceutical agents,
an alcohol,
a fatty acid, and
the shell comprising:
gelatin, and
at least one plasticizing agent,
wherein said shell has an initial state and an equilibrium state, wherein said initial state shell is underplasticized and said fill contains an alcohol amount in excess of an alcohol amount necessary to solubilize said pharmaceutical agent and said alcohol is not present in the initial state shell composition, and wherein said equilibrium state said shell becomes plasticized by the alcohol from said fill to provide suitable capsule hardness and said fill retains sufficient alcohol to maintain said pharmaceutical agent in solution.

2. The soft elastic capsule of claim 1 wherein the amount of alcohol present in said fill in initial state is in the range of 100-200% greater than the amount necessary to solubilize said pharmaceutical agent.

3. The soft elastic capsule of claim 1 wherein a portion in the range of 40-50% of the amount of alcohol present in said fill in initial state migrates into the shell upon equilibrium state.

4. The soft elastic capsule of claim 1 wherein said pharmaceutical agent comprises a solubilized HIV inhibiting compound or a combination of solubilized HIV inhibiting compounds.

5. The soft elastic capsule of claim 4 wherein said HIV inhibiting compound is (2S, 3S, 5S)-5-(N-((N-methyl-N-N-((2-isopropyl-4-thiazolyl)-methyl)amino)carbonyl)-L-valinyl)amino-2-(N-((5-thiazolyl)methoxy-carbonyl)-amino-1,6-diphenyl-3-hydroxyhexane (ritonavir).

6. The soft elastic capsule of claim 4 wherein said combination of solubilized HIV inhibiting compounds is (2S, 3S, 5S)-5-(N-((N-methyl-N-N-((2-isopropyl-4-thiazolyl)-methyl)amino)carbonyl)-L-valinyl)amino-2-(N-((5-thiazolyl)methoxy-carbonyl)-amino-1,6-diphenyl-3-hydroxyhexane (ritonavir) and (2S, 3S, 5S)-2-(2,6 dimethylphenoxyacetyl)-amino-3-hydroxy-5-(2S-(1-tetrahydropyrimid-2-onyl)-3-methyl-butanoyl)amino-1,6-diphenylhexane (lopinavir).

7. The soft elastic capsule of claim 4 wherein said solubilized HIV inhibiting compound or said combination of solubilized HIV inhibiting compounds is present in said fill in a range of 10-35% by weight.

8. The soft elastic capsule of claim 1 wherein said alcohol is propylene glycol or ethanol.

9. The soft elastic capsule of claim 8 wherein propylene glycol or ethanol are present in said fill in initial state in a range of 3-20% by weight.

10. The soft elastic capsule of claim 1 wherein said alcohol concentration in the fill decreases by not more than 20% after reaching said equilibrium state.

11. The soft elastic capsule of claim 10 wherein after reaching said equilibrium state said shell has acceptable physical characteristics.

12. The soft elastic capsule of claim 11 wherein the acceptable physical characteristics of said shell comprise
a hardness in the range of about 4.5 to about 9 Newtons,
a total weight of said capsule within 95% of initial weight of capsule and total weight of said fill within 85% of initial weight of said fill,
a moisture content of not more than 2% of said fill weight of said capsule,
a disintegration time of not more than 30 minutes, and
an alcohol concentration of at least 40 mg per gram of said fill.

13. The soft elastic capsule of claim 1 wherein said fatty acid is oleic acid.

14. The soft elastic capsule of claim 13 wherein said oleic acid is present in said fill composition in initial state in the range of 50-80% by weight.

15. The soft elastic capsule of claim 1 wherein said fill further comprises a surfactant.

16. The soft elastic capsule of claim 15 wherein said surfactant is Polyoxyl 35 castor oil (Cremophor EL®).

17. The soft elastic capsule of claim 15 wherein said surfactant is present in the range of 0-10% by weight of the fill.

18. The soft elastic capsule of claim 1 wherein the gelatin in said capsule is present in the range of 30-60% by weight of the shell.

19. The soft elastic capsule of claim 1 wherein the plasticizing agent is sorbitol, glycerin, macrogols, or Andrisorb™.

20. The soft elastic capsule of claim 19 wherein the plasticizing agent is present in initial state in the range of 4-18% by weight of the shell.

21. The soft elastic capsule of claim 1 wherein the ratio of the weight percentage of gelatin to plasticizing agent at said equilibrium state is in the range of 2:1 to 4:1.

22. The soft elastic capsule of claim 1 wherein the fill in an initial state comprises:
is (2S, 3S, 5S)-5-(N-(N-((N-methyl-N-N-((2-isopropyl-4-thiazolyl)-methyl)amino)carbonyl)-L-valinyl)amino-2-(N-((5-thiazolyl)methoxy-carbonyl)-amino-1,6-diphenyl-3-hydroxyhexane (ritonavir) and (2S, 3S, 5S)-2-(2,6 dimethylphenoxyacetyl)-amino-3-hydroxy-5-(2S-(1-tetrahydropyrimid-2-onyl)-3-methyl-butanoyl)amino-1,6-diphenylhexane (lopinavir) in the range of 10-35% by weight of the fill composition;
polyethylene glycol in the range of 3-20% by weight of the fill composition;
oleic acid in the range of 50-80% by weight of the fill; and wherein the shell in an initial state comprises:
gelatin in the range of 30-60% by weight of the shell, and Andrisorb in the range of 4-18% by weight of the shell.

23. A method for making soft elastic capsules containing a pharmaceutical agent or a combination of pharmaceutical agents, the method comprising the steps of:
preparing a fill composition wherein the fill composition comprises
a pharmaceutical agent or combination of pharmaceutical agents,
a fill alcohol, and
a fatty acid;
preparing a shell composition wherein the shell composition comprises
gelatin, and
at least one plasticizing compound;
forming an soft elastic capsule wherein said shell has an initial state and an equilibrium state, wherein said initial state shell is underplasticized and said fill contains an alcohol amount in excess of an alcohol amount necessary to solubilize said pharmaceutical agent and said alcohol is not present in the initial state shell composition; and
equilibrating said soft elastic capsule wherein equilibrium state said shell becomes plasticized by the alcohol from said fill to provide suitable capsule hardness and said fill retains sufficient alcohol to maintain said pharmaceutical agent in solution.

24. The method of claim 23 wherein said pharmaceutical agent comprises a solubilized HIV inhibiting compound or a combination of solubilized HIV inhibiting compounds.

25. The method of claim 23 wherein said a]cohol is propylene glycol or ethanol.

26. The method of claim 23 wherein said fatty acid is oleic acid.

27. The method of claim 23 wherein said fill further comprises a surfactant.

28. The method of claim 23 wherein the plasticizing agent is sorbitol, glycerin, macrogols, or Andrisorb™.
